(19) United States
(12) Patent Application Publication
(10) Pub. No.: US 2010/0273247 A1
(43) Pub. Date: Oct. 28, 2010

(54) SEMI-QUANTITATIVE IMMUNOLOGICAL DETECTION DEVICE WITH DIFFERENTIAL ANTIBODIES OR SUBSTRATES

(75) Inventors: Yo Han Choi, Daejeon (KR); Monn Youn Jung, Daejeon (KR); Seon Hee Park, Daejeon (KR)

Correspondence Address: AMPACC Law Group, 3500 188th Street S.W., Suite 103, Lynnwood, WA, 98037, US

(73) Assignee: Electronics and Telecommunications Research Institute, Daejeon (KR)

(21) Appl. No.: 12/746,997

(22) PCT Filed: Jun. 24, 2008
(86) PCT No.: PCT/KR08/03585

§ 371 Date: Jun. 9, 2010

(30) Foreign Application Priority Data
Dec. 11, 2007 (KR) 10-2007-0128231

(51) Int. Cl. C12M 1/34

(52) U.S. Cl. 435/287.2

(57) ABSTRACT

A semi-quantitative immunological detection device with differential antibodies and substrates is provided. The semi-quantitative immunological detection device with differential antibodies, includes: a sample injecting portion through which a sample is injected; and a reaction portion where a plurality of reacting materials reacting with an MUT (material under test) contained in the sample with different sensitivities to generate marker materials are disposed in the same space. Accordingly, a diagnosis result can be visually identified with user's eyes, and the diagnosis result similar to that of a quantitative assay apparatus can be provided.

### TECHNICAL FIELD

The present invention relates to an immunological detection device, and more particularly, to a semi-quantitative immunological detection device with differential antibodies and substrates, capable of being visually identified with user's eyes and providing a diagnosis result similar to that of a quantitative assay apparatus.

This work was supported by the IT R&D program of MIC/IITA [2006-S-007-02, Ubiquitous Health Monitoring Module and System Development]

### BACKGROUND ART

Recently, various strip-shaped diagnosis kits for determining presence of a specific protein through a reaction between an antigen and an antibody have been widely used. The strip-shaped diagnosis kits are medical products that are very familiar to un-trained persons.

The most representative product is a pregnancy test kit for testing pregnancy-associated hormones. In addition, various diagnosis kits for testing heart disease or prostate cancer have been commercialized.

Such conventional strip-shaped diagnosis kits provide on/off-type information or a quantitative digit as a diagnosis result.

In case of an on/off-type strip, the diagnosis result can be visually identified with eyes without an additional assay apparatus, so that the on/off-type strip can be simply used with a low cost. However, if a detection signal is unclear, a user has to select one of the irreconcilable diagnosis results according to user's personal experience.

In addition, the on/off-type strip cannot be used for a case where a diagnosis result is not suitably expressed by on/off-type information.

On the other hand, in case of a strip-shaped diagnosis kit providing a quantitative digit as a diagnosis result, the problem of the on/off-type strip can be solved. However, since an expensive assay apparatus is additionally needed, this diagnosis kit cannot be easily used by general persons.

### DISCLOSURE OF INVENTION

### Technical Problem

As described above, in case of a conventional on/off-type strip, a diagnosis result can be visually identified with eyes without an additional assay apparatus, so that the on/off-type strip can be used simply with a low cost. However, an error of diagnosis may occur according to user's emotional state. In particular, when a boundary line between on and off states is unclear, the on/off-type strip cannot be used. On the other hand, in case of a conventional diagnosis kit providing a quantitative digit as a diagnosis result, an expensive assay apparatus is additionally needed, so that this diagnosis kit cannot be easily used by general persons.

The present invention provides a semi-quantitative immunological detection device for semi-quantitatively expressing an existence range of an MUT (material under test) beyond an on/off concept without an assay apparatus, capable of visually identifying a diagnosis result and providing the diagnosis result similar to that of a quantitative assay apparatus and a method of manufacturing the semi-quantitative immunological detection device.

### TECHNICAL SOLUTION

According to an aspect of the present invention, there is provided a semi-quantitative immunological detection device with differential antibodies, comprising: a sample injecting portion through which a sample is injected; and a reaction portion where a plurality of reacting materials reacting with an MUT (material under test) contained in the sample with different sensitivities to generate marker materials are disposed in the same space.

In the above aspect of the present invention, the reacting materials may be made of antibodies having different affinities to the MUT. In addition, the reacting materials may be made of different amounts of an antibody having the same affinity to the MUT.

In addition, the reacting materials may be disposed in parallel to each other with respect to the sample injecting portion. In addition, the reacting materials may be disposed in series in the order of reaction sensitivities with respect to the sample injecting portion.

In addition, the semi-quantitative immunological detection device may further comprise a sample applying portion which applies the sample injected through the sample injecting portion to the reaction portion. In addition, the sample applying portion may be implemented with a filter or a micro channel.

According to another aspect of the present invention, there is provided a semi-quantitative immunological detection device with differential antibodies or substrates, comprising: a sample injecting portion through which a sample is injected; and a plurality of reaction portions which are separately disposed in independent spaces and react with an MUT (material under test) contained in the sample with different sensitivities to generate marker materials.

In the above aspect of the present invention, antibodies having different affinities to the MUT may be fixedly disposed in the respective reaction portions. In addition, different amounts of antibodies having the same affinities to the MUT may be fixedly disposed in the respective reaction portions. In addition, each of the reaction portions may comprise: a signal detecting portion where an equal amount of the same reacting material is fixedly disposed; and a substrate storing portion which supplies one of substrates having different reaction sensitivities to the MUT to the signal detecting portion.

In addition, the reaction portions may be disposed in parallel to each other with respect to the sample injecting portion. In addition, the reaction portions may be disposed in series in the order of reaction sensitivities with respect to the sample injecting portion.

In addition, the semi-quantitative immunological detection device may further comprise a sample applying portion which applies the sample injected through the sample injecting portion to the reaction portion. In addition, the sample applying portion may be implemented with a filter or a micro channel.

### ADVANTAGEOUS EFFECTS

In a semi-quantitative immunological detection device with differential antibodies and substrates according to the present invention, a concentration of an MUT can be semi-quantitatively expressed beyond an on/off concept without an additional assay apparatus, so that a diagnosis result can be visually identified with user's eyes and the diagnosis result similar to that of a quantitative assay apparatus can be provided.

Accordingly, it is possible for general users to easily perform self test with a low cost. In addition, in case of small and medium -sized medical facilities that cannot easily utilize an expensive assay apparatus, it is possible to simply perform an initial diagnosis process with a low cost by using the apparatus according to the present invention.

### BRIEF DESCRIPTION OF THE DRAWINGS

FIGS. 1 and 2 are schematic views illustrating a semi-quantitative immunological detection device using differential antibodies according to an embodiment of the present invention.

FIGS. 3 and 4 are schematic views illustrating a semi-quantitative immunological detection device using differential antibodies according to another embodiment of the present invention.

FIG. 5 is a schematic view illustrating a semi-quantitative immunological detection device using differential antibodies according to still another embodiment of the present invention.

### BEST MODE FOR CARRYING OUT THE INVENTION

Hereinafter, embodiments of the present invention will be described in detail with reference to the accompanying drawings. For clarifying the present invention, detailed description of well-known functions and constructions will be omitted.

In the accompanying drawing, elements having similar functions and operations are denoted by the same reference numerals.

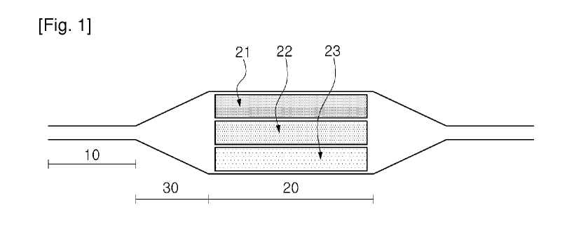

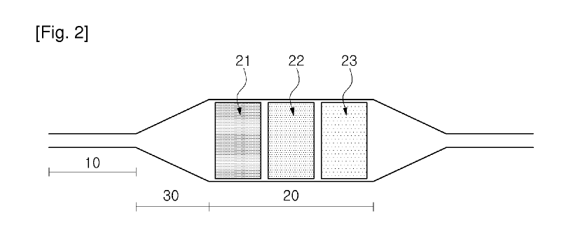

FIGS. 1 and 2 are schematic views illustrating a semi-quantitative immunological detection device using differential antibodies according to an embodiment of the present invention.

As shown in FIGS. 1 and 2, the semi-quantitative immunological detection device includes a sample injecting portion 10 through which a sample of a mixture of a signal-detection antibody and a material under test(MUT) is injected, a reaction portion 20 where a plurality of reacting materials 21, 22, and 23 reacting with the MUT (contained in the sample) with different reaction sensitivities to generate marker materials are disposed in the same space, and a sample applying portion 30 which applies the sample (injected through the sample injecting portion 10) to the reaction portion 20.

As shown in FIG. 1, the reacting materials 21, 22, and 23 may be disposed in parallel to each other with respect to the sample injecting portion 10. Alternatively, as shown in FIG. 2, the reacting materials 21, 22, and 23 may be disposed in series in the order of reaction sensitivities with respect to the sample injecting portion 10.

The reacting materials 21, 22, and 23 may be made of different antibodies having different affinities to the same MUT. Alternatively, the reacting materials 21, 22, and 23 may be made of different amounts of the same antibody having the same affinity to the same MUT.

The sample applying portion 30 may be implemented with a filter for applying the sample to the reaction portion 20 by percolating the sample.

Alternatively, the sample applying portion 30 may be implemented with a micro channel for applying the sample to the reaction portion 20 by using a predetermined physical force such a pressure difference or a capillary force.

In the semi-quantitative immunological detection device having the aforementioned construction, concentrations of the reaction materials can be semi-quantitatively detected through the following operations.

Firstly, when a sample of a mixture of an MUT and a signal-detection antibody is injected into a sample injecting portion 10, the sample applying portion 30 applies the sample to the reaction portion 20.

While the signal-detection antibody attached with the MUT passes through the reaction portion 20, the signal-detection antibody is attached to the reacting materials 21, 22, and 23 by using the MUT as a mediator. The reacting materials 21, 22, and 23 are fixedly disposed in the reaction portion 20 in advance.

Next, when a substrate is decomposed through a function of an enzyme such as AP (alkaline phosphatase) or HRP (horseradish peroxidase) conjugated to the signal-detection antibody, a marker material (for example, a colorant) which can be visually identified with user's eyes is generated.

If a large amount of the MUT is contained in the sample, that is, if the MUT have a high concentration, all the reacting materials 21, 22, and 23 are reacted, so that the corresponding marker materials are generated.

If the MUT has a middle concentration, only the predetermined reacting materials (for example, 21 and 22) having middle and highest affinities are reacted, so that the corresponding marker materials are generated.

If the MUT has a concentration less than a predetermined concentration, only the predetermined reacting material (for example, 21) having the highest affinity or the largest amount thereof is reacted, so that the corresponding marker material is generated.

Therefore, a user can visually identify the occurrence of the marker materials, so that it is possible to directly check the concentration of the MUT that is to be currently analyzed.

In the above embodiment, an enzyme is used as an example of the signal-detection antibody. Alternatively, an antibody coupled with a gold nano particle or the like may be used, if needed. Moreover, any material capable of performing the same function can be used as the signal-detection antibody.

In the immunological detection device shown in FIG. 1, a method of aligning the reacting materials according to the affinities is not specifically limited. However, in the immunological detection device shown in FIG. 2, a reacting material having the highest affinity is disposed at a position where the sample is firstly contacted, that is, a position closer to the sample injecting portion, and reacting materials having the lower affinities are sequentially disposed.

In this case, in the region where the antibody having the highest concentration is disposed, even a lowest concentration of the MUT can be detected as a signal. In the region where the antibody having the lowest concentration is disposed, only the highest concentration of the MUT can be detected as a signal.

In the above embodiment, three types of antibodies and three levels of concentration are used. However, it should be noted that the types of antibodies and the levels of concentration can be further differentiated according to properties of the MUT and necessity of detailed diagnosis results.

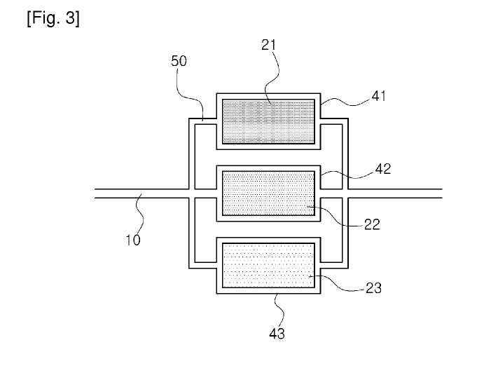

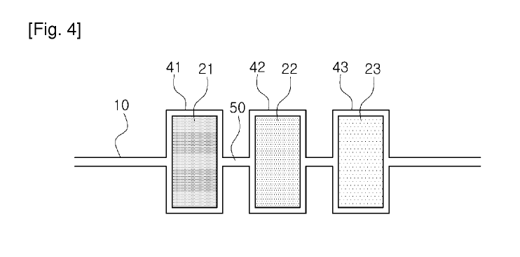

FIGS. 3 and 4 are schematic views illustrating a semi-quantitative immunological detection device using differential antibodies according to another embodiment of the present invention.

As shown in FIGS. 3 and 4, the semi-quantitative immunological detection device includes a sample injecting portion 10 through which a sample of a mixture of a signal-detection antibody and an MUT is injected, a plurality of reaction portions 41, 42, and 43 which are separately disposed in independent spaces and in which reacting materials 21, 22, and 23 reacting with the MUT (contained in the sample) with different sensitivities to generate marker materials are fixedly disposed, and a sample applying portion 50 which applies the sample (injected through the sample injecting portion 10) to the reaction portions 41, 42, and 43.

As shown in FIG. 3, the reaction portions 41, 42, and 43 may be disposed in parallel to each other with respect to the sample injecting portion 10. Alternatively, as shown in FIG. 4, the reaction portions 41, 42, and 43 may be disposed in series in the order of reaction sensitivities with respect to the sample injecting portion 10.

The reaction portions 41, 42, and 43 may be implemented with different antibodies having different affinities to the same MUT. Alternatively, the reaction portions 41, 42, and 43 may be implemented with different amounts of the same antibody having the same affinity to the same MUT.

The sample applying portion 50 may be implemented with a filter for applying the sample to the reaction portions 41, 42, and 43 by percolating the sample. Alternatively, the sample applying portion 50 may be implemented with a micro channel for applying the sample to the reaction portions 41, 42, and 43 by using a predetermined physical force such a pressure difference or a capillary force.

Therefore, in the semi-quantitative immunological detection device having the constructions shown in FIGS. 3 and 4, a plurality of reacting materials 21, 22, and 23 are spatially separated from each other, and after that, concentrations of the reacting materials can be semi-quantitatively detected through the operations shown in FIGS. 1 and 2.

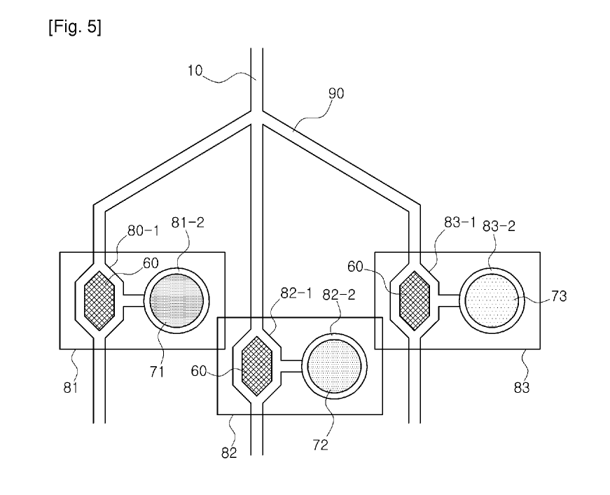

FIG. 5 is a schematic view illustrating a semi-quantitative immunological detection device using differential antibodies according to still another embodiment of the present invention.

As shown in FIG. 5, the semi-quantitative immunological detection device includes a sample injecting portion 10 through which a sample of a mixture of a signal-detection antibody and an MUT is injected, a plurality of reaction portions 81, 82, and 83 which are separately disposed in independent spaces and which reacts with the MUT (contained in the sample) with different sensitivities to generate marker materials, and a sample applying portion which applies the sample (injected through the sample injecting portion 10) to the reaction portions 81, 82, and 83.

Each of the reaction portions 81, 82, and 83 includes a corresponding one of signal detecting portions 81-1, 82-1, and 83-1 where the equal amount of the same reacting material 60 is fixedly disposed and a corresponding one of substrate storing portions 81-2, 82-2, and 83-2 which supplies a corresponding one of substrates 71, 72, and 73 having different reaction sensitivities to the same MUT to the corresponding one of the signal detecting portions 81-1, 82-1, and 83-1.

In FIG. 5, the reaction portions 81, 82, and 83 are disposed in parallel to each other with respect to the sample injecting portion 10. Alternatively, the reaction portions 81, 82, and 83 may be disposed in series in the order of reaction sensitivities with respect to the sample injecting portion 10, if needed.

The sample applying portion 90 may be implemented with a filter for applying the sample to the reaction portions 81, 82, and 83 by percolating the sample. Alternatively, the sample applying portion 90 may be implemented with a micro channel for applying the sample to the reaction portions 81, 82, and 83 by using a predetermined physical force such a pressure difference or a capillary force.

In the semi-quantitative immunological detection device having the aforementioned construction, concentrations of the reaction materials can be semi-quantitatively detected through the following operations.

Firstly, when a sample of a mixture of an MUT and a signal-detection antibody is injected into a sample injecting portion 10, the sample applying portion 90 applies the sample to the reaction portions 81, 82, and 83.

While the signal-detection antibody attached with the MUT passes through each of the signal detecting portions 81-1, 82-1, and 83-1 of the reaction portions 81, 82, and 83, the signal-detection antibody is attached to the reacting material 60 by using the MUT as a mediator. The reacting material 60 is fixedly disposed in each of the signal detecting portions 81-1, 82-1, and 83-1 in advance.

The substrate storing portion 81-2, 82-2, and 83-2 that store substrates having different reaction sensitivities to the MUT apply the substrates to the signal detecting portions 81-1, 82-1, and 83-1, respectively, so as to generate corresponding signals. When the substrates are decomposed through a function of an enzyme such as AP or HRP conjugated to the signal-detection antibodies in the signal detecting portions 81-1, 82-1, and 83-1, corresponding marker materials are generated.

If the MUT has a concentration higher than a predetermined concentration, all the signal detecting portions 81-1, 82-1, and 83-1 generate marker materials corresponding to all the substrates. If the MUT has a middle concentration, only the signal detecting portions 81-1 and 82-1 generate marker materials corresponding to the substrate having highest reaction sensitivity and the substrate having a middle reaction sensitivity. If the MUT has a concentration lower than another predetermined concentration, only the signal detecting portion 81-1 generates a marker material corresponding to the substrate having the highest reaction sensitivity.

Therefore, a user can visually identify the occurrence of the marker materials, so that it is possible to semi-quantitatively detect signals similarly to the aforementioned embodiments of the present invention.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A semi-quantitative immunological detection device with differential antibodies, comprising:
a sample injecting portion through which a sample is injected; and
a reaction portion where a plurality of reacting materials reacting with an MUT (material under test) contained in the sample with different sensitivities to generate marker materials are disposed in the same space.

2. The semi-quantitative immunological detection device of claim 1, wherein the reacting materials are made of antibodies having different affinities to the MUT.

3. The semi-quantitative immunological detection device of claim 1, wherein the reacting materials are made of different amounts of an antibody having the same affinity to the MUT.

4. The semi-quantitative immunological detection device of claim 1, wherein the reacting materials are disposed in parallel to each other with respect to the sample injecting portion.

5. The semi-quantitative immunological detection device of claim 1, wherein the reacting materials are disposed in series in the order of reaction sensitivities with respect to the sample injecting portion.

6. The semi-quantitative immunological detection device of claim 5, wherein the reacting material having the highest reaction sensitivity is disposed to be closest to the sample injecting portion, and the reacting material having the lowest reaction sensitivity is disposed to be farthest from the sample injecting portion.

7. The semi-quantitative immunological detection device of claim 1, further comprising a sample applying portion which applies the sample injected through the sample injecting portion to the reaction portion.

8. The semi-quantitative immunological detection device of claim 7, wherein the sample applying portion is implemented with a filter or a micro channel.

9. A semi-quantitative immunological detection device with differential antibodies or substrates, comprising:
a sample injecting portion through which a sample is injected; and
a plurality of reaction portions which are separately disposed in independent spaces and react with an MUT (material under test) contained in the sample with different sensitivities to generate marker materials.

10. The semi-quantitative immunological detection device of claim 9, wherein antibodies having different affinities to the MUT are fixedly disposed in the respective reaction portions.

11. The semi-quantitative immunological detection device of claim 9, wherein different amounts of antibodies having the same affinities to the MUT are fixedly disposed in the respective reaction portions.

12. The semi-quantitative immunological detection device of claim 9, wherein each of the reaction portions comprises:
a signal detecting portion where an equal amount of the same reacting material is fixedly disposed; and
a substrate storing portion which supplies one of substrates having different reaction sensitivities to the MUT to the signal detecting portion.

13. The semi-quantitative immunological detection device of claim 9, wherein the reaction portions are disposed in parallel to each other with respect to the sample injecting portion.

14. The semi-quantitative immunological detection device of claim 9, wherein the reaction portions are disposed in series in the order of reaction sensitivities with respect to the sample injecting portion.

15. The semi-quantitative immunological detection device of claim 9, further comprising a sample applying portion which applies the sample injected through the sample injecting portion to the reaction portion.

16. The semi-quantitative immunological detection device of claim 15, wherein the sample applying portion is implemented with a filter or a micro channel.
